(19) United States
(12) Patent Application Publication
(10) Pub. No.: US 2025/0170325 A1
(43) Pub. Date: May 29, 2025

(54) UNIVERSAL HOLDER DEVICE FOR ANESTHESIA AND CRITICAL CARE/INTENSIVE CARE UNIT PATIENT MANAGEMENT

(71) Applicant: Gabriel G. Alvarez, Coral Gables, FL (US)

(72) Inventor: Gabriel G. Alvarez, Coral Gables, FL (US)

(21) Appl. No.: 18/104,989

(22) Filed: Feb. 2, 2023

Related U.S. Application Data
(60) Provisional application No. 63/325,651, filed on Mar. 31, 2022.

(51) Int. Cl. A61M 5/14; A61M 16/08

(52) U.S. Cl. CPC A61M 5/1417; A61M 5/1408; A61M 2209/082

(57) ABSTRACT

The present invention is directed to a universal holder for safely supporting a number of anesthesia and critical care equipment in an organized manner on a single device. The universal holder includes a principal structure that includes an upper portion, a middle portion defining a notched channel, and a lower portion. A receiver plate is movably mounted and is vertically adjustable within the notched channel of the main plate. The receiver plate includes a structure for mounting pressure monitoring devices and manifolds. A removable transesophageal echocardiogram “TEE” probe mount is provided and is removably attachable to the receiver plate to secure the TEE probe to the universal holder. The universal holder includes a self-adjusting flexible hook to attach the universal holder to a bed handrail of a hospital bed and a foldable base plate to support the universal holder between a mattress and the frame of the hospital bed.

### CROSS-REFERENCE TO RELATED APPLICATIONS

This application claims the benefit of U.S. Provisional Patent Application Ser. No. 63/325,651, filed on Mar. 31, 2022, which is incorporated herein in its entirety.

### FIELD OF THE INVENTION

The present invention relates generally to medical equipment holders, and more particularly, to a universal holder for safely supporting, securing and organizing a variety of anesthesia and critical care equipment on a single device.

### BACKGROUND OF THE INVENTION

During a variety of medical and/or surgical procedures it is necessary to place a patient under anesthesia to enable performing the procedure, optimize conditions for the surgeon and for the comfort and safety of the patient. In order to do so, it is necessary to utilize a number of different types of anesthesia equipment and accessories. Such equipment may include an invasive pressure monitoring system to monitor the patient's heart and blood pressure, breathing circuits to maintain proper oxygenation and ventilation to the patient and various types of manifolds with multiple ports to control the flow of the medications and hydrating fluids into the heart and blood stream of the patient.

Complications can arise in trying to safely position, organize and secure all the necessary anesthesia equipment in a relatively small area around the head of the surgical operating table supporting the patient and at the intensive care unit critical care bed. For example, the use of invasive pressure monitoring systems having a plurality of pressure transducers is a critical and necessary piece of equipment to monitor a patient's regional and systemic blood pressures. The invasive pressure transducers are typically mounted on a vertical rectangular plate attached to a vertical metal pole or similar structure. The pole and the plate are each and independently, height adjustable to position level to hold and “set” the pressure transducer(s) at a “heart level” of a patient to sample the pressures at the only correct position—at level “CERO”. Put differently, the invasive pressure transducers need to be always “calibrated” at the level of the heart. Any other level position (other than “CERO”) will lead to pressure sampling errors and patient mismanagement. The invasive pressure transducer system (vertical pole, vertical plate and transducers) is attached to the lateral rail of the surgical operating table, which warranties an approximate height of the transducers to be at “heart level” following any height (up or down) change of the operating table. This approximate height level is less accurate if the surgical operating table changes to a steep incline position, head down or head up. The invasive pressure transducer(s) vertical plate can also be free-standing, tedder and unsecured from and over the surgical operating table or ICU bed or attached to a separate structure. In this case, every time the position of the surgical operating table or the hospital ICU bed is adjusted to any position (height or level of inclination), the position of the pressure transducer(s) needs to be continuously readjusted to maintain the pressure transducer at the correct heart level. Failure to do this will cause significant sampling errors, misinterpretations, and mismanagement. Lastly, each separate component of the invasive pressure transducer system (metal clips, vertical pole and vertical plate) may be missing at the time of urgent/emergency assembly or malfunction or brake, disabling the whole system.

The administration of anesthesia also requires the use of an anesthesia breathing circuit composed by a long corrugated breathing tube, filters and varied diameter union connections, including one to the endotracheal tube of the patient. The breathing circuit is typically supported and secured on a “tree” type holder structure that is held and stabilized under the surgical mattress of the operating table. Any change of the surgical operating table position, (height, level of inclination) the “tree” holder structure will move with it while securing the breathing circuit to prevent any tension on the union connections or prevent kinks that may cause a sudden obstruction. Without the tree type holder structure any position change of the operating table, especially extreme inclinations or side rotations needed for robotic surgery, will cause undue tension at multiple points of the breathing circuit, including the vital connection with the endotracheal tube. Furthermore, the malposition or misuse of the tree holder structure can be the cause of endotracheal tube dislodgement despite appropriate means to secure it.

Additionally, complex surgeries such as open cardiac surgery or hybrid surgical procedures such as minimal invasive cardiac procedures with radiology and ultrasound imaging, typically require the use of a transesophageal echocardiography or “TEE.” This continuous imaging modality requires an ultrasound probe which is inserted through the mouth into the esophagus behind the heart. The US probe has a control handle to manipulate various features, including distal maneuvering of the probe like flexion/extension. Thus this control handle has a ring attached to use as a “hanger” to any desired structure which usually is located on a separate intravenous pole adjacent to the surgical operating table. The control handle is typically mounted on a fork clamp holder or a self-made soft aluminum stylet hook or taped to an intravenous pole. Again, if the OR surgical operating table is moved in any direction too far from the control handle when is mounted not in use by the practitioner, the probe will withdraw from the esophagus and completely dislodge from the patient's mouth, which is often difficult to reinsert during the ongoing surgical procedure already with the sterile drapes barrier on and over the patients head.

Critically ill patients requiring surgery and postoperative care in the Intensive Care Unit, often depends upon multiple life-supporting medications at once. It is not uncommon to have limited venous access points to the patient, having the need to use manifolds with multiple ports as a standard to facilitate the delivery of drugs, usually through an indwelling central venous line (CVL) catheter going directly to the heart with a point of entry at the neck. There are many types of manifold designs; nevertheless, all of them can cause significant tension by pulling the CVL when connected to it with all and each of the ports in used with multiple medication lines coming from medical pumps. To resolve this problem and prevent the major complication of suddenly pulling out the CVL, the manifolds are taped to the surgical table mattress, which is ineffective after the tape becomes wet or due to the hours of tension its subjected-to. Also, the manifolds may be secured by an additional clamp mounted on an adjacent IV pole which adds to the overall cost.

In addition to each individual limitation of the above anesthesia equipment relating the patient's position on the surgical table or critical care bed, then, if conjoint together, the risk of interfering or becoming entangled with each other causes further complications.

Accordingly, there is a need for solutions to the aforementioned problems. For instance, there is an established need for a single device that can efficiently and effectively organize and maintain all the necessary anesthesia equipment in a central position close to the head of the patient on the surgical operating table or the ICU hospital bed. There is a further established need for a single device that can be attached to a hospital bed or surgical operating table and is automatically adjustable to compensate for any movement of the hospital ICU bed or surgical operating table to maintain the transducers in the correct position relative to a patient's heart level and to prevent the equipment from becoming dislodged from the patient's trachea and/or esophagus during the procedure.

### SUMMARY OF THE INVENTION

The present invention is directed to a universal holder for safely supporting a number of anesthesia and critical care equipment in an organized manner on a single device. The universal holder includes a principal structure or main vertical plate having an upper or top portion, a center or middle portion defining a notched channel and a lower portion. Another vertical receiver plate perpendicular to the longitudinal axis of the main plate is movably mounted on the middle portion of the main plate and is vertically adjustable within the notched channel of the main plate to a desired height. The receiver plate includes a plurality of structures for mounting pressure transducers and manifolds. A posterior latch mechanism is provided to secure the receiver plate in a vertical position on the main plate. A transesophageal echocardiogram “TEE” probe mount is provided and is removably attachable to the posterior side of the receiver plate to secure the TEE probe control handle to the universal holder. The universal holder includes a self-adjusting rotational hook to attach the universal holder to a hospital transport stretcher bed and intensive care unit bed. The universal holder also includes a foldable base plate to support and secure the universal holder between the mattress and the frame of the operating surgical table. The upper portion of the main plate includes lateral arms defining gaps for securing breathing circuits, while the bottom portion of the main plate includes projections to hang pressure infusion bags on the universal holder.

In a first implementation of the invention, a universal holder for safely supporting and securing a variety of anesthesia and critical care equipment on a single device is provided, the universal holder comprising:
a principal structure defining a notched channel having multiple projections extending into said channel to define a plurality of opposed gaps therebetween in the principal structure;
a receiver plate mounted for vertical movement on said principal structure and having multiple brackets for attachment of invasive pressure transducers;
a latch mechanism mounted on said receiver plate and engageable within said gaps defined between said multiple projections of said principal structure to reliably secure said receiver plate in a vertical position on said principal structure;
   a self-adjusting arm rotatably mounted to said principal structure; and
a base plate pivotally attached to said principal structure, said base plate moveable between a raised condition substantially adjacent to said principal structure and a lowered condition extending substantially perpendicular to said principal structure to a horizontal position.

In a second aspect, the receiver plate may be mounted perpendicular to the longitudinal axis of the principal structure.

In another aspect, the principal structure may include a first front side and a second back side, the receiver plate being mounted on first front side of the principal structure.

In another aspect, the latch mechanism may be mounted on the second back side of the principal structure.

In another aspect, the latch mechanism may include a housing mounted to the receiver plate and a latch arm movable relative to the housing, the latch arm being laterally movable into and out of the gaps in the notched channel to secure the receiver plate at a desire vertical location on the principal structure.

In another aspect, the receiver plate may include at least one hook for support of a manifold.

In another aspect, the receiver plate may include at least one sliding pocket cup for support of a manifold.

In another aspect, the receiver plate may include at least one bracket for releasable retention of a pressure transducer.

In another aspect, the at least one bracket may be located on a front side of the receiver plate.

In another aspect, the at least one bracket may be located on a back side of the receiver plate.

In another aspect, the self-adjusting rotational arm may include a flexible curved or inverted L-shaped hook holder for engagement with a bed handrail.

In another aspect, the self-adjusting arm may be rotatable through a range of +/−45° relative to the principal structure.

In another aspect, the universal holder further may include a transesophageal echocardiogram probe handle holder.

In another aspect, the transesophageal echocardiogram probe handle holder may be removably mounted on the receiver plate.

In another aspect, the transesophageal echocardiogram (“TEE”) probe handle holder may include a backing plate removably attachable to the receiver plate and a C-clip extending from the backing plate.

In another aspect, the top portion of the principal structure may include a lateral arm defining a gap for support of a breathing hose.

In another aspect, the bottom portion of the principal structure may include at least one hook for hanging a pressurized infusion bag.

In a second implementation of the invention, a method for safely supporting and securing a variety of anesthesia and critical care equipment on a single device is provided, the method comprising:
providing a universal holder including a principal structure defining a notched channel having multiple projections extending into the channel to define a plurality of opposed gaps therebetween in the principal structure;
a receiver vertical plate mounted for vertical movement on the principal structure and having multiple bracket holder clips for attachment of invasive pressure transducers;
securing the principal structure to a surgical operating table;
mounting a pressure transducer on the receiver plate;
mounting a manifold on the receiver plate; and
mounting at least one infusion bag on the principal structure.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a front plan view of a universal holder for anesthesia and critical care equipment in accordance with an illustrative embodiment of the present invention and mounted on a hospital bed handrail;

FIG. 2 presents a back side isometric view of the universal holder for anesthesia and critical care equipment illustrated in FIG. 1;

FIG. 3 presents a side plan view, partially shown in section, of the universal holder for anesthesia and critical care equipment, with a base of the universal holder being folded up, in use on the hospital bed handrail;

FIG. 4 presents a back plane view with a hook of the universal holder for anesthesia and critical care equipment rotated with the bed handrail;

FIG. 5 presents a front side isometric view of a universal holder for anesthesia and critical care equipment with equipment attached;

FIG. 6 presents an isometric view of the universal holder for anesthesia and critical care equipment mounted on the handrail of a hospital transport stretcher bed;

FIG. 7 presents a front plane view of the universal holder mounted on the surgical operating table with the base plate inserted between a mattress and a frame of the surgical operating table;

FIG. 8 presents a side plane view, partially shown in section, of the universal holder mounted on the surgical operating table with the base plate positioned between the mattress and the frame of the surgical operating table; and

FIG. 9 presents an isometric view of the universal holder for anesthesia and critical care equipment mounted on the hospital transport stretcher bed of FIG. 8.

Like reference numerals refer to like parts throughout the several views of the drawings.

### DETAILED DESCRIPTION

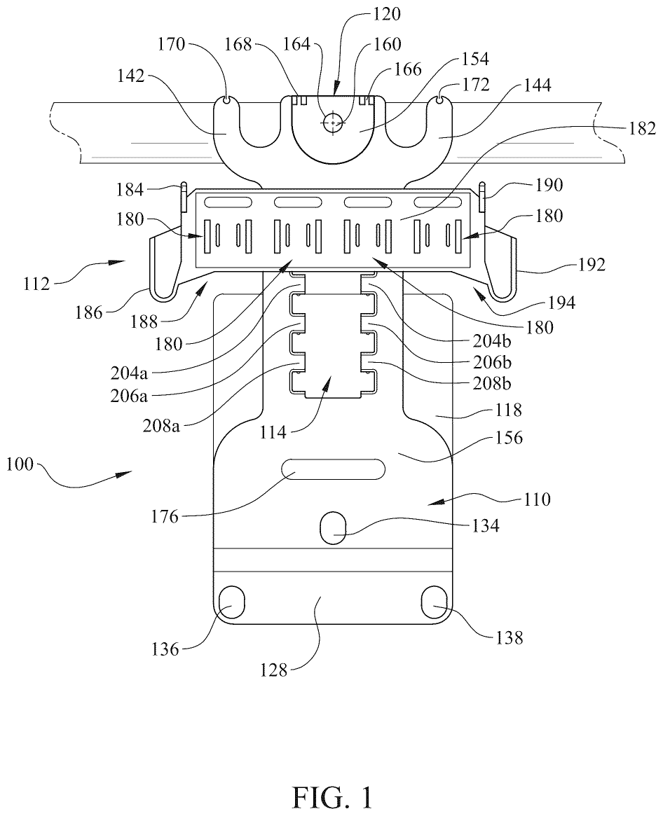

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “right”, “front”, “back”, “rear”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a universal holder for anesthesia and critical care equipment to safely and in an organized manner hold and secure a variety of medical equipment and devices used by an anesthesiologist during a surgical procedure and the critical care team in the Intensive Care Unit.

As used herein, the term “substantial” is a degree or result acceptable within the medical field.

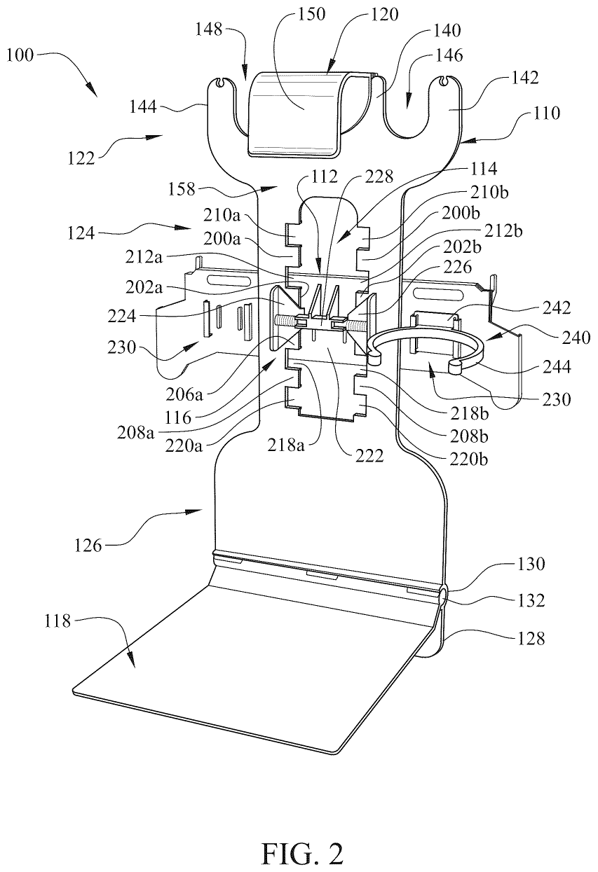

Referring to FIGS. 1-5, a universal holder for a variety of anesthesia equipment used during a surgical procedure, hereinafter universal holder 100, is illustrated in accordance with an exemplary embodiment of the present invention. The universal holder 100 generally includes a vertical principal structure or main vertical plate 110 and a universal transducer and manifold holder or horizontal receiver plate 112 movably mounted on the main vertical plate 110. The horizontal receiver plate 112 is mounted for vertical movement on the main vertical plate 110 to adjust the height of the horizontal receiver plate 112, and thus equipment mounted thereon, relative to a patient's heart level as discussed in more detail hereinbelow. More specifically, the main vertical plate 110 defines a notched channel 114 and the horizontal receiver plate 112 is height adjustable on the main vertical plate 110 to definitive set heights in the notched channel 114. A latch mechanism 116 (FIG. 2) is affixed to the horizontal receiver plate 112 and is engageable with the main vertical plate 110 at various distinct locations within the notched channel 114 of the main vertical plate 110 to secure the horizontal receiver plate 112 the various heights relative to the main vertical plate 110.

The disclosed universal holder 100 is designed to be attached to a rail of a hospital transport stretcher bed, critical intensive care unit bed or a surgical operating table and includes a foldable base plate 118 and a self-adjusting flexible hook 120. The foldable base plate 118 is pivotally mounted to the main vertical plate 110 and when extended horizontally is provided for insertion between the frame and the mattress of the surgical hospital table to hold the universal holder 100 adjacent to the patient head at heart level and secure to surgical operating table as described in more detail hereinbelow. The self-adjusting hook or arm 120 is rotatably mounted on the main vertical plate 110 and is provided to be attached to a handrail of a hospital transport stretcher bed or critical intensive care unit bed. The self-adjusting arm 120 rotates relative to the main vertical plate 110 as the handrail moves with the back or upper body portion of the hospital bed, when is raised and lowered in an incline angle. This allows the horizontal receiver plate 112, mounted on the main vertical plate 110, to remain leveled in a proper position relative to a heart of a patient positioned on the hospital bed as also discussed in more detail hereinbelow.

As noted hereinabove, the universal holder 100 is provided to hold a variety of necessary medical anesthesia equipment in a safe and organized manner during a surgical procedure and postoperative care in the critical intensive care unit. For example, the universal holder 100 is designed to primarily hold the horizontal receiver plate 112 to receive invasive pressure transducers 510, a variety of manifolds 800, vital safety sensitive equipment such as breathing circuits 600 and a transesophageal echocardiogram probe handle 700 (not shown). The universal holder 100 may additionally hold multiple infusion pressurized bags as part of the medical equipment needed for invasive pressure monitoring of the patient.

For example, as shown, the horizontal receiver plate 112 contains a plurality of clips 180 as receiver docks for pressure transducers 510 having associated stopcocks 512 with pressurized fluid tubes 514. The breathing circuits 600 may include at least one corrugated breathing hose 610. The variety of manifolds 800, for example, manifold 800 includes separate ports 812 attached to a backing or mounting plate 814 and a tubes or hoses 816 extending between and from the ports 812. The infusion bags 900 include pressure bags 910 having support loops 912 for mounting the pressure bags 912 on the universal holder as discussed hereinbelow.

The main vertical plate 110 is the primary vertical support for the horizontal receiver plate 112 and generally includes a top portion 122 mounting the self-adjusting rotating arm 120, a middle portion 124 defining the notched channel 114 and mounting the horizontal receiver plate 112 and a bottom portion 126 mounting the horizontal base plate 118 when extended. The main vertical plate 110 further includes an extension plate 128 extending from the bottom portion 126 of the main vertical plate 110. A pivot tube 130 is formed between the bottom portion 126 of the main vertical plate 110 and the extension plate 128 of the main vertical plate 110.

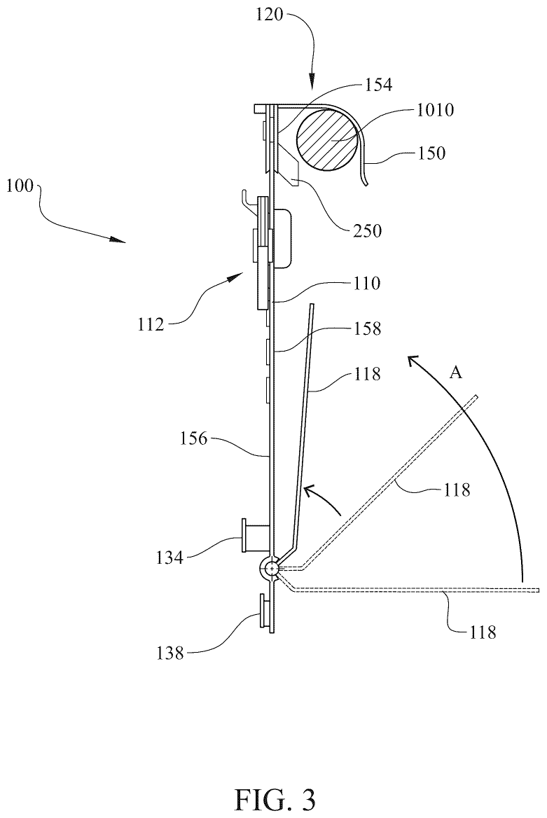

As noted hereinabove, the base plate 118 is pivotally mounted to the main vertical plate 110. This allows the base plate 118 to be folded up against the main vertical plate 110 when not in use (see FIG. 3) and folded down to a position substantially perpendicular to the main vertical plate 110 when used to support the main vertical plate 110 on the surgical operating table. The base plate 118 includes a pivot pin 132 rotatably mounted within the pivot tube 130 of the main vertical plate 110. While not specifically shown, the pivot pin 132 of the base plate 118 and the pivot tube 130 of the main vertical plate 110 include cooperating structure to maintain the base plate 118 perpendicular to the main vertical plate 110 when the base plate 118 is in the folded down in horizontal position.

As shown, the bottom portion 126 of the main vertical plate 110 includes a projecting medial hook 134 extending outward from the bottom portion 126. The medial hook 134 is provided to hold a pressurized infusion bag similar to infusion bags 900. The extension plate 128 also has a pair of projecting lateral hooks 136 and 138 to support the infusion bags 900. Specifically, the lateral hooks 136 and 138 of the extension plate 128 of the main vertical plate 110 are provided to receive the support loops 912 of the pressurized infusion bags 900.

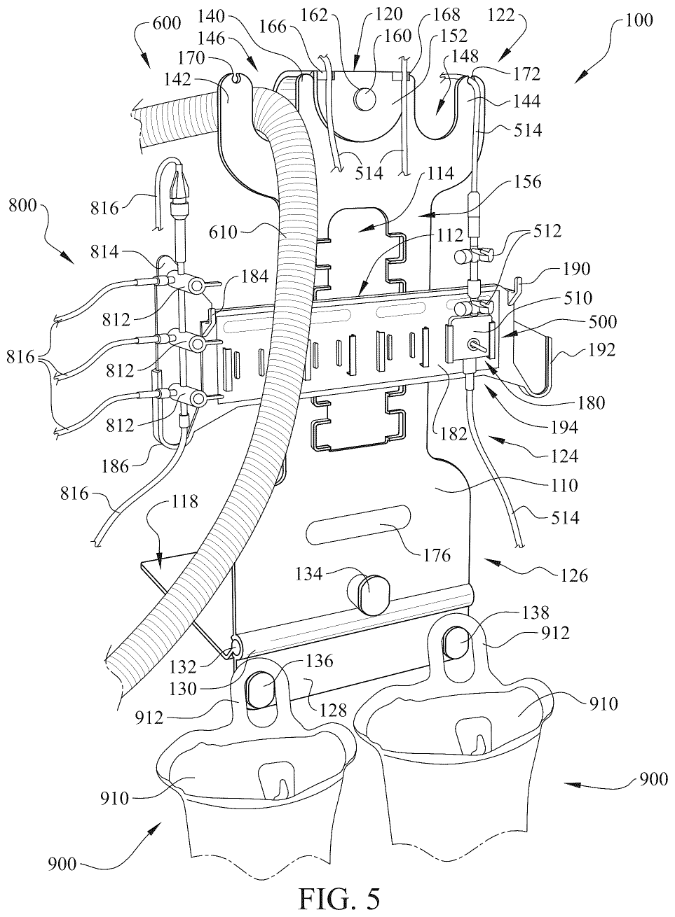

Referring now to FIGS. 2 and 5, the top portion 122 of the main vertical plate 110 includes a center portion 140, mounting the self-adjusting rotating hook 120, and a pair of lateral arms 142 and 144 extending laterally and upward from the center portion 140. The lateral arms 142 and 144 defined respective gaps 146 and 148 between the lateral arms 142 and 144 and the center portion 140 for support of the breathing hoses 610 of the breathing circuit 600. By positioning the breathing hose 610 of the breathing circuit 600 within one of the gaps 146 or 148 of the top portion 122 of the main vertical plate 110, the breathing hose 610 is stabilized, secured and prevented from pulling free of a patient during an operation.

Referring to FIGS. 1-5, the self-adjusting rotational arm 120 is designed to be attached to a hospital transport stretcher and intensive care unit bed handrail and is provided to allow the main vertical plate 110, and thus the horizontal receiver plate 112 of the universal holder 100 to remain vertical and in proper alignment level with a patient's heart as the back or upper portion of the hospital bed is raised in inclination from cero (0) to seventy five (75) degrees upwards while under the strain of gravity. The self-adjusting arm 120, in one exemplary embodiment, generally includes a flexible inverted L-shaped arm 150 (FIG. 2), a front plate 152 (FIG. 5) and a back plate 154 (FIG. 1). The flexible arm 150 is provided to hang from the hospital transport stretcher bed handrail or snap pressed onto the critical care unit bed handrail and capture the top portion of the handrail between the flexible arm 150 and the back plate 154 of the self-adjusting hook 120. The front plate 152 of the self-adjusting hook 120 is positioned adjacent a front side 156 (FIG. 5) of the main plate 110 and the back plate 154 of the self-adjusting hook 120 is positioned adjacent to a rear side 158 (FIG. 2) of the main vertical plate 110. A rotation shaft 160 extends through the center portion 140 of the top portion 122 of the main vertical plate 110 and rotatably supports the self-adjusting hook 120 thereon. Specifically, the rotation shaft 160 extends through a hole 162 formed in the front plate 152 of the self-adjusting hook 120 and a hole 164 formed through the back plate 154 of the self-adjusting hook 120 to allow the self-adjusting hook 120 to rotate relative to the main vertical plate 110 and the horizontal receiver plate 112 of the universal holder 100.

In order to secure various tubes, such as, for example, pressurized fluid tubes 514 of the pressure monitoring devices 500, the self-adjusting hook 120 is provided with one or more notches 166, 168, etc. (FIGS. 2 and 5) designed to grip the tubes 514 and prevent any slippage thereof. Similarly, the lateral arms 142 and 144 of the top portion 122 of the main vertical plate 110 are provided with notches 170 and 172, respectively, to secure various pressurized fluid tubes associated with the anesthesia and critical care equipment.

As best shown in FIGS. 1 and 5, the front side 156 of the main vertical plate 110 of the universal holder 100 may be provided with indicia 176. The indicia 176 may be in the form of product name, manufacture name, etc.

With continued reference to FIGS. 1 and 5, and as noted hereinabove, the horizontal receiver plate 112 of the universal holder 100 is provided to securely hold and organize various devices such as for example, the pressure transducers devices 500, the transesophageal echocardiogram probe control panel handle (not shown) and the various manifolds 800. The horizontal receiver plate 112 is provided with a plurality of holder clips 180, which act as receiver docks, formed on a front side 182 of the horizontal receiver plate 112. The holder clips 180 are provided to securely and releasably hold the pressure transducers 510 of the pressure monitoring devices 500.

Additionally, the horizontal receiver plate 112 of the universal holder 100 further includes a hook 184 and a sliding pocket or cup 186 at a first end 188 of the horizontal receiver plate 112 to hold a manifold 800. As shown in FIG. 5, the mounting of a type of manifold 800 is secured behind the hook 184 and sliding into the pocket to rests in the cup 186. A second hook 190 and a second cup 192 are provided on a second end 194 of the horizontal receiver plate 112 to hold an additional manifold 800 if necessary.

With specific reference now to FIG. 2, and as noted hereinabove, the horizontal receiver plate 112 of the universal holder 100 is vertically adjustable relative to the main vertical plate 110 of the universal holder 100. The main vertical plate 110 includes a plurality of pairs of opposed projections 200a, 200b, 202a, 202b, 204a, 204b, 206a, 206b and 208a and 208b extending into the notched channel 114 defined in the center portion 140 of the main vertical plate 110. The plurality of pairs of opposed projections 200a, 200b, 202a, 202b, 204a, 204b, 206a, 206b and 208a and 208b create top gaps 210a and 210b, middle gaps 212a, 212b, 214a, 214b, 216a, 216b, 218a, 218b and bottom gaps 220a and 220b in the center portion 140 of the main vertical plate 110. Gaps 214a and b and gaps 216a and b are hidden from view by the horizontal receiver plate 112.

The latch mechanism 116, attached to a back side 222 of the horizontal receiver plate 112, is designed to engage these gaps between the disclosed projections to secure the horizontal receiver plate 112 in its vertical position at the desire height level on the main vertical plate 110. The latch mechanism 116 includes laterally slidable latch arms 224 and 226 and a central spring housing 228. The spring housing 228 houses a biasing spring (not shown) for biasing the latch arms 224 and 226 outwardly from the spring housing 228.

In operation, the latch arms 224 and 226 of the latch mechanism 116 are moved inwardly against the bias of the spring in the spring housing 228 to draw the latch arms 224 and 226 out of the gaps between the disclosed projections extending into the notched channel 114 in the main vertical plate 110. The horizontal receiver plate 112 can then be vertically moved relative to the main vertical plate 110 to the proper height and the latch arms 224 and 226 released. The bias of the spring in the spring housing 228 moves and secures the latch arms 224 and 226 outwardly into one of the pairs of gaps i.e., the top gaps 210a and 210b, middle gaps 212a, 212b, 214a, 214b, 216a, 216b, 218a, 218b or bottom gaps 220a and 220b in the center portion 140 of the main vertical plate 110, to secure the horizontal receiver plate 112 in position on the main vertical plate 110.

As shown, the horizontal receiver plate 112 includes two additional holder clips 230 formed on the back side 222 of the horizontal receiver plate 112. Additional pressure transducers 510 can be thus mounted on the back side 222 of the horizontal receiver plate 112.

The universal holder 100 additionally include a removable transesophageal echocardiogram (“TEE”) probe holder 240 which is configured to be mounted in the holder clips 180 on the front side 182 of the horizontal receiver plate 112 or in the holder clips 230 on the back side 222 of the horizontal receiver plate 112. The TEE probe holder 240 includes a backing plate 242 and an enlarged flexible C-clip 244 extending from the backing plate 242. The backing plate 242 of the TEE probe holder 240 is releasably engageable with the holder clips 180 and 230 and the C-clip 244 is provided to releasably hold a TEE Probe (not shown).

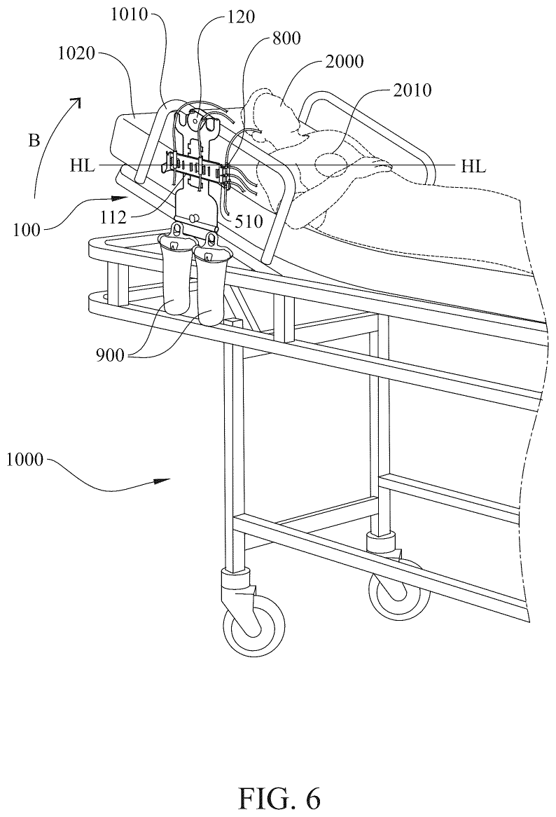

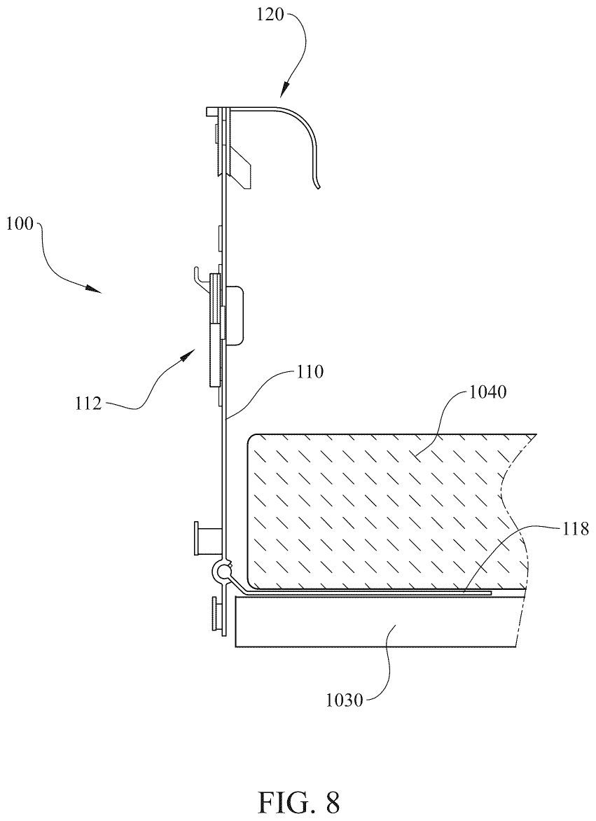

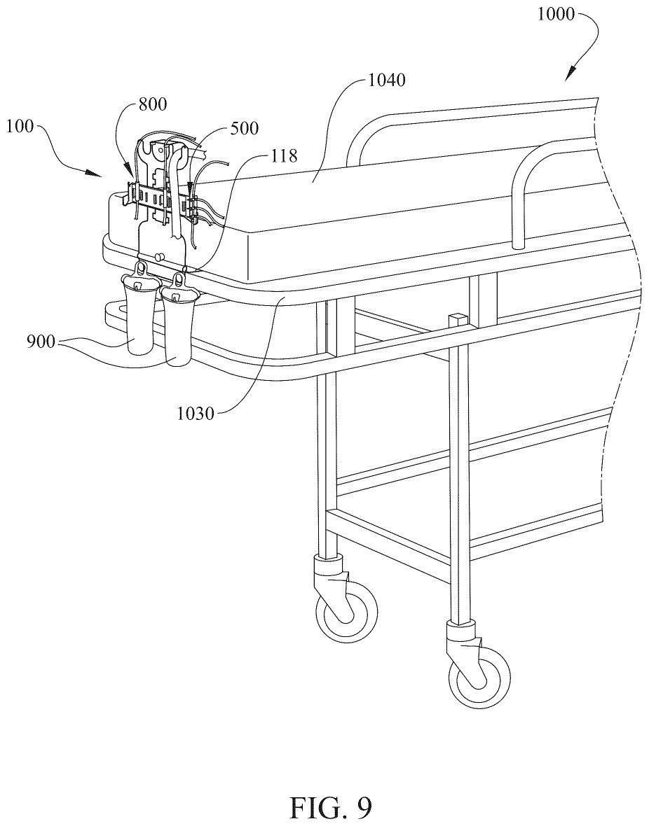

Referring now to FIGS. 3-9, the use of the universal holder 100 to support anesthesia equipment on a hospital transport stretcher bed such as, for example generic hospital bed 1000 (FIG. 6), a critical intensive care unit bed or surgical operating table will now be described. While the following description is given with regard to securing the universal holder 100 to a generic hospital bed 1000, it is understood that the universal holder can be similarly used with and attached to a surgical operating table or critical intensive care unit bed.

With reference to FIGS. 3-6, in a first method of using the universal holder 100, the self-adjusting hook 120 of the universal holder 100 is attached to a hospital bed handrail 1010 of the hospital bed 100. Specifically, as best shown in FIG. 3, the flexible arm 150 of the self-adjusting hook 120 is snapped over the handrail 1010 of the hospital bed 1000 securing the handrail 1010 between the flexible arm 150 and the back plate 154 of the self-adjusting hook 120. As shown, the self-adjusting hook 120 additionally include a leg 250 to position and stabilize the self-adjusting hook 120 on the hospital bed handrail 1010.

Before or after attaching the self-adjusting hook 120 of the universal holder 100 to the handrail 1010 of the hospital bed 1000, the base plate 118 is pivoted upward in the direction of arrow “A” (FIG. 3) to position the base plate 118 adjacent to the rear side 158 of the main vertical plate 110. This moves the base plate 118 out of the way and allows the universal holder 100 to hang vertically from the hospital bed handrail 1010 from the self-adjusting hook 120. Once the universal holder 100 has been properly attached to the hospital bed handrail 1010, and the base plate 118 pivoted out of the way, the horizontal receiver plate 112 can be moved to the desired vertical position on the main vertical plate 110 and secured in position with the latch mechanism 116 as described hereinabove.

With continued reference for the moment to FIG. 3, it can be seen that the medial hook 134 extends further outwardly from the front side 154 of the main vertical plate 110 than do the lateral hooks 136 and 138, see for example lateral plate 158. This prevents any interference between adjacent infusion bags 900 (FIGS. 5 and 6) hanging therefrom.

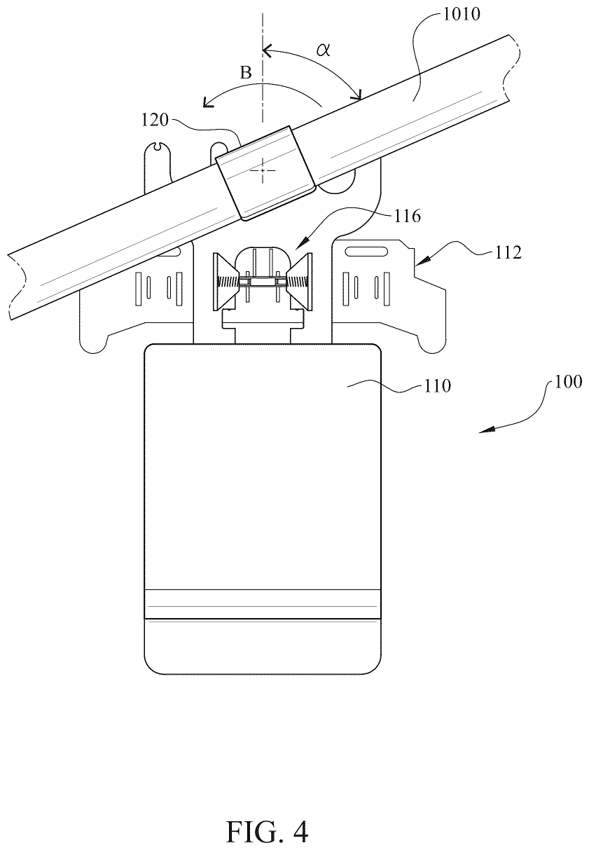

Turning now to FIGS. 4-6, and as noted hereinabove, the disclosed universal holder 100 maintains pressure monitoring devices 500, and in particular the pressure transducers 510 (FIG. 6) of the pressure monitoring devices 500, in proper horizontal position relative to a patient's 2000 heart 2010 regardless of the positioning of an upper body portion 1020 of the hospital bed 1000.

As shown in FIGS. 4 and 6, as the upper body portion 1020 of the hospital bed 1000 is raised in the direction of arrow “B”, the self-adjusting hook 120 of the universal holder 100 moves with the handrail 1010 (FIG. 4) while the main vertical plate 110 and horizontal receiver plate 112 of the universal holder 100 remain in a vertical condition relative to the handrail 1010. The self-adjusting hook 120 has a range of motion relative to the main vertical plate 110 through an angle \acute{α} of approximately +/−45°. As best shown in FIG. 6, this allows the horizontal receiver plate 112, and thus any pressure transducers 510 attached thereto, to remain on the correct heart level plane “HL” with respect to the patient's 2000 heart 2010 thereby avoiding any pressure sampling errors.

Once the upper body portion 1020 of the hospital bed 1000 has been raised to the desired position, the other anesthesia equipment, for example the infusion bags 900 can be attached to the universal holder 100. The manifolds 800 may be similarly attached to the horizontal receiver plate 112 of the universal holder 100 or may be prepositioned thereon. While not specifically shown, the breathing circuits 600 including the breathing hose 610 is attached to the universal holder 100 as described hereinabove, and a TEE probe control handle (not shown) may be held on the universal holder 100 with the TEE holder 240 (FIG. 2).

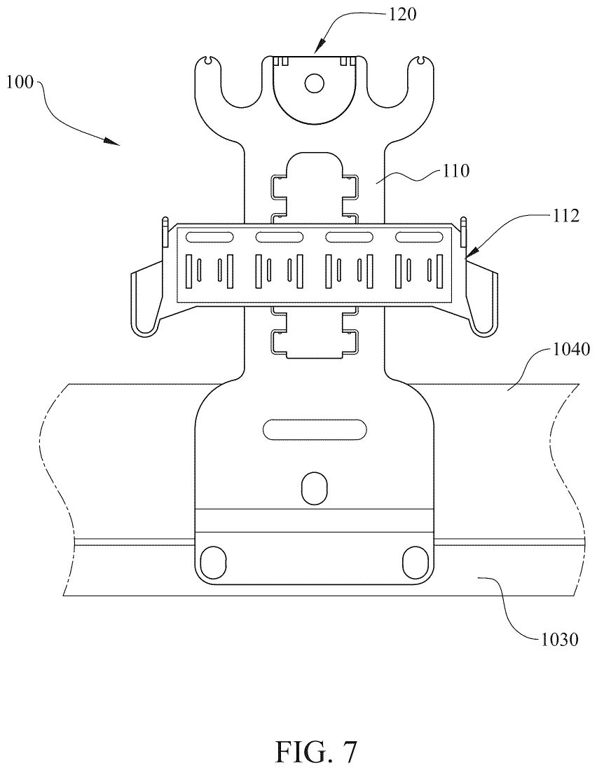

Referring to FIGS. 7-9, in an alternative method of attaching the universal holder 100 to the hospital bed 1000, the base plate 118 is moved to the folded down position extending perpendicularly to the main plate 100 (FIG. 3) and the base plate 118 inserted between a frame 1030 of the hospital bed 1000 and a body support member (i.e., mattress or other covering 1040) of the hospital bed 1000. In this method of securing the universal holder 100 to the hospital bed 1000, the self-adjusting hook 120 is not used. The horizontal receiver plate 112 is adjusted to the proper height relative to the main plate 110 and the patient's heart (not shown) and secured there with the use of the latch mechanism 116. Thereafter, the anesthesia equipment, such as, for example, the pressure monitoring devices 500, the breathing circuits 600 (FIG. 6), the TEE probe (not shown), the manifolds 800 and the infusion bags 900 are attached to the universal holder 100 as described hereinabove.

Thus, the disclosed universal holder 100 provides a single point of attachment for various equipment used in the administration of anesthesia to a patient to keep and maintain the equipment in a safe and organized manner. The universal holder 100 further provides a novel and adjustable method of maintaining pressure monitoring equipment at the correct level relative to a patient's heart regardless of the positioning of the hospital bed.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A universal holder for safely supporting and securing a variety of anesthesia and critical care equipment on a single device, comprising:
a principal structure defining a notched channel having multiple projections extending into the channel to define a plurality of opposed gaps therebetween;
a receiver plate movably mounted on the principal structure and having multiple holder clips for attachment of medical equipment;
a latch mechanism mounted on the receiver plate and engageable within the gaps defined between the multiple projections of the principal structure for securing the receiver plate on the principal structure;
a self-adjusting arm rotatably mounted to the principal structure; and
a base plate pivotally attached to the principal structure.

2. The universal holder of claim 1, wherein the base plate is moveable between a raised condition substantially adjacent to the principal structure and a lowered condition extending substantially perpendicular to the principal structure.

3. The universal holder of claim 1, wherein the receiver plate is mounted perpendicular to the longitudinal axis of the principal structure and configured to move vertically within the notched channel.

4. The universal holder of claim 1, wherein the principal structure includes a front side and a back side, and the receiver plate is mounted on the front side of the principal structure.

5. The universal holder of claim 1, wherein the latch mechanism includes a housing mounted to the receiver plate and a latch arm movable relative to the housing.

6. The universal holder of claim 1, wherein the receiver plate includes at least one hook for support of a manifold.

7. The universal holder of claim 1, wherein the receiver plate includes at least one pocket cup for support of a manifold.

8. The universal holder of claim 1, wherein the receiver plate includes at least one bracket for releasable retention of a pressure transducer.

9. The universal holder of claim 1, wherein the self-adjustable arm includes a curved-shaped arm portion for engagement with a bed handrail.

10. The universal holder of claim 9, wherein the self-adjustable arm rotates to maintain the principal structure substantially neutral to a vertical axis.

11. The universal holder of claim 1, wherein the self-adjustable arm is rotatable through a range of motion of about +/−45 degrees relative to the principal structure.

12. The universal holder of claim 1, further comprises a removable TEE probe holder.

13. The universal holder of claim 12, wherein the TEE probe holder includes a backing plate removably attachable to the receiver plate and a probe receiving head portion extending from the backing plate.

14. The universal holder of claim 1, wherein a top portion of the principal structure includes a lateral arm defining a gap for support of a breathing hose.

15. The universal holder of claim 1, wherein a bottom portion of the principal structure includes one or more hooks for holding one or more pressurized infusion bags.

16. The universal holder of claim 1, wherein the base plate is disposable between a bed support frame and a body support member and configured to maintain the principal structure in an upright position.

17. A universal holder for safely supporting and securing a variety of anesthesia and critical care equipment on a single device, comprising:
a principal structure defining a notched channel having multiple projections extending into the channel to define a plurality of opposed gaps therebetween;
a receiver plate mounted for vertical movement on the principal structure and having multiple holder clips for attachment of medical equipment;
a latch mechanism mounted on the receiver plate and engageable within the gaps defined between the multiple projections of the principal structure to reliably secure the receiver plate in a vertical position on the principal structure;
a self-adjusting arm rotatably mounted to the principal structure; and
a base plate pivotally attached to the principal structure, the base plate moveable between a raised condition substantially adjacent to the principal structure and a lowered condition extending substantially perpendicular to the principal structure.

18. The universal holder of claim 17, wherein the latch mechanism includes a housing mounted to the receiver plate and a latch arm movable relative to the housing, the latch arm configured for lateral movement into and out of the gaps in the notched channel to secure the receiver plate at a desirable vertical position on the principal structure.

19. The universal holder of claim 17, wherein the self-adjustable arm rotates to maintain the receiver plate level with a patient's heart level plane as the principal structure hangs from the bed handrail.

20. A universal holder for safely supporting and securing a variety of anesthesia and critical care equipment on a single device, comprising:
a principal structure including a top portion, a bottom portion, and a central portion defining a notched channel having multiple projections extending into the channel to define a plurality of opposed gaps therebetween;
a receiver plate mounted for vertical movement on the principal structure and having multiple holder clips for attachment of anesthesia and critical care equipment;
a latch mechanism mounted on the receiver plate and engageable within the gaps defined between the multiple projections of the principal structure to reliably secure the receiver plate in a vertical position on the principal structure;
a removable TEE probe holder including a backing plate removably attachable to the receiver plate and a probe receiving head portion extending from the backing plate;
a self-adjusting arm rotatably mounted to the principal structure,
wherein the self-adjustable arm engages with a bed handrail, and is rotatable through a range of motion of about +/−45 degrees relative to the principal structure to maintain the principal structure substantially normal to a horizontal plane as the principal structure hangs from the bed handrail; and
a base plate pivotally attached to the principal structure, the base plate moveable between a raised condition substantially adjacent to the principal structure and a lowered condition extending substantially perpendicular to the principal structure.
